(19) United States
(12) Patent Application Publication
(10) Pub. No.: US 2007/0106392 A1
(43) Pub. Date: May 10, 2007

(54) Acetabular cup locking mechanism

(75) Inventors: Damon Servidio, Towaco, NJ (US); Imants Liepins, Bloomingburg, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/268,963

(22) Filed: Nov. 8, 2005

(51) Int. Cl. A61F 2/34

(52) U.S. Cl. 623/22.28

(57) ABSTRACT

A prosthetic cup assembly is disclosed. The assembly preferably includes a shell portion, a metallic insert and a polymeric insert. The cooperation between each of the inserts and the shell portion is preferably such that alignment of the components is assured and movement between the inserts and the shell portion is substantially impossible.

### BACKGROUND OF THE INVENTION

The present invention relates to acetabular cup replacement assemblies, and more particularly to an acetabular cup replacement assembly which includes a single shell that allows for the interchangeability between metallic/ceramic and polyethylene inserts.

Total hip replacement surgery typically entails the removal and replacement of the femoral head portion of the femur, as well as the resurfacing and replacement of the acetabular cup. In both cases, prosthetic implants are utilized to replace the removed bone portions. Although these types of surgeries have become rather common place, surgeons are often faced with decisions during surgery relating to the particular prosthetic implants utilized, in addition to their respective orientation and positioning. For example, acetabular cup replacements may require a surgeon to first implant a shell portion and thereafter select different cup inserts and position them with respect to the shell portion. Several attempts at providing beneficial acetabular cup assemblies have been developed heretofore. Two examples of such acetabular cup assembly designs are disclosed in U.S. Pat. Nos. 6,475,243 and 6,610,097, the disclosures of which are hereby incorporated by reference herein.

Acetabular cup assemblies do indeed provide benefits to surgeons, but they are not without their own drawbacks. In certain designs, surgeons utilizing and implanting such shell and insert combinations often have a difficult time ensuring that the two components are properly aligned. Frequently, the relatively small working space which is available to a surgeon during a hip replacement prevents the complete view of the replacement components. The previously designed acetabular cup replacements simply have not offered a solution to this problem. In addition, in certain other designs, the mating of the shell and insert portions of acetabular cup replacements is such that movements of the two components, although best described as micro-movements, can develop over time. This may be caused by differences in the tolerances of the two components, and is clearly not desired for long term implantation into a patient.

Therefore, there exists a need for an acetabular cup replacement assembly which allows for easy assembly and alignment in situ, and provides a substantially non-moveable coupling between a shell portion and insert.

### SUMMARY OF THE INVENTION

A first aspect of the present invention is a prosthetic cup assembly. A first embodiment of this assembly may include a metallic shell having an outer shell surface and an inner shell surface, the outer shell surface being adapted for insertion into the acetabulum of a patient and the inner shell surface including a plurality of scallops and a female taper. The assembly may also include a metallic insert having an outer insert surface, the outer insert surface being adapted for insertion into the inner shell surface of the shell, the outer insert surface including a plurality of lobes adapted for engagement with the scallops and a male taper adapted for engagement with the female taper. Preferably, the dimensional relationship between the insert and the shell is such that the insert may be inserted into the shell from an axially aligned position or from an angled position, and insertion of the outer insert surface within the inner shell surface allows fixable attachment of the insert to the shell.

In accordance with this first aspect of the present invention, the shell may be constructed of many different materials including cobalt chrome alloys, stainless steel and titanium. In one preferred embodiment, the shell may include twelve scallops and the insert may include twelve lobes. The shell may further include a circumferential groove, twelve diametrical recesses and at least one barb. The insert may further include twelve rim portions and a centralizing chamfer. Still further, the outer shell surface may include bone growth inducing surfaces. The insert may be inserted into the shell in situ.

A second aspect of the present invention is another prosthetic cup assembly. In accordance with one embodiment of this second aspect, the assembly may include a metallic shell having an outer shell surface and an inner shell surface, the outer shell surface being adapted for insertion into the acetabulum of a patient and the inner shell surface including a plurality of scallops, a circumferential groove and at least one barb. The assembly may further include a polymeric insert having an outer insert surface, the outer insert surface being adapted for insertion into the inner shell surface of the shell, the outer insert surface including a plurality of lobes, a circumferential bead and a plurality of stepped sections. Preferably, the plurality of lobes are adapted for engagement with the scallops, the circumferential bead is adapted for engagement with the circumferential groove, and the at least one barb are adapted for engagement with the plurality of stepped sections upon insertion of the outer insert surface within the inner shell surface, thereby fixable attaching the insert to the shell.

Yet another aspect of the present invention is a kit for use in hip replacement surgery. The kit may include at least one shell, the shell having an outer shell surface and an inner shell surface, the outer shell surface being adapted for insertion into the acetabulum of a patient and the inner shell surface including a plurality of scallops, a female taper, a circumferential groove and at least one barb. The kit may further include at least one metallic insert having an outer metallic insert surface being adapted for insertion into the inner shell surface of the shell, the outer metallic insert surface including a plurality of lobes and a male taper, where the plurality of lobes are adapted for engagement with the scallops and the male taper is adapted for engagement with the female taper upon insertion of the outer insert surface within the inner shell surface, thereby fixably attaching the insert to the shell. The kit may also include at least one polymeric insert having an outer insert surface being adapted for insertion into the inner shell surface of the shell, the outer insert surface including a plurality of lobes, a circumferential bead and a plurality of stepped sections, where the plurality of lobes are adapted for engagement with the scallops, the circumferential bead is adapted for engagement with the circumferential groove, and the at least one barb are adapted for engagement with the plurality of stepped sections upon insertion of the outer insert surface within the inner shell surface, thereby fixably attaching the insert to the shell.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a perspective view of an acetabular cup replacement assembly in accordance with the present invention.

FIG. 2 is a top perspective view of a shell portion of the acetabular cup replacement assembly of FIG. 1.

FIG. 3 is a side perspective view of a metallic/ceramic insert of the acetabular cup replacement assembly of FIG. 1.

FIG. 4 is a side perspective view of a polymeric insert of the acetabular cup replacement assembly of FIG. 1.

FIG. 5 is a side perspective view of the metallic/ceramic insert of FIG. 3 in one angular orientation with respect to the shell portion of FIG. 2.

FIG. 6 is a side cross sectional view of the metallic/ceramic insert of FIG. 3 in another angular orientation with respect to the shell portion of FIG. 2.

FIGS. 7a-7d are side cross sectional views of the metallic/ceramic insert of FIG. 3 being inserted into the shell portion of FIG. 2 from yet another angular orientation.

FIG. 8 is an enlarged view of the shell portion of FIG. 2, showing a barb located in the interior of the shell portion.

FIG. 9 is an enlarged view of the polymeric insert of FIG. 4, showing a step located on the exterior surface of the insert.

FIG. 10 is a cross sectional view of the polymeric insert of FIG. 4 partially located within the shell portion of FIG. 2, just prior to engagement.

FIGS. 11a-11d are enlarged cross sectional views of the mating portions of the shell portion of FIG. 2 and the polymeric insert of FIG. 4, in sequential mating positions.

FIG. 12 is a cross sectional view of the shell portion of FIG. 2 with the polymeric insert of FIG. 4 attached thereto.

### DETAILED DESCRIPTION

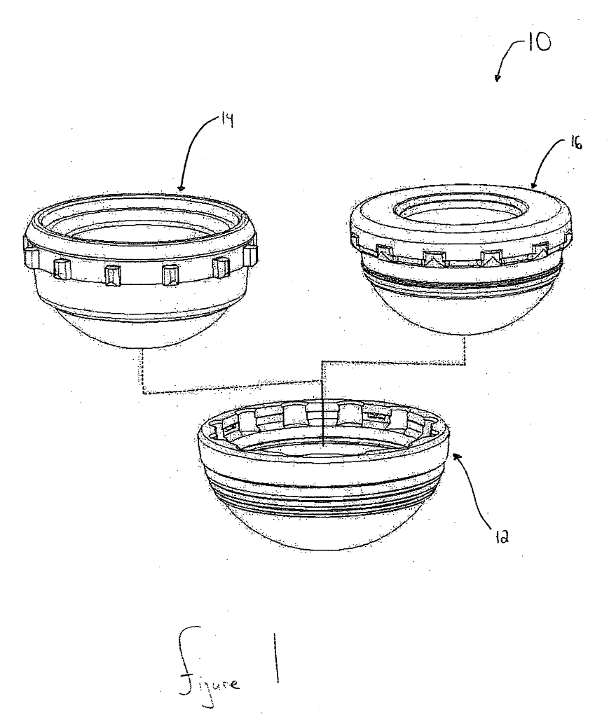

Referring to the drawings, wherein like reference numerals refer to like elements, there is shown in FIG. 1, an acetabular cup replacement assembly designated generally by reference numeral 10. As shown in that figure, assembly 10 includes a shell portion 12 and a metallic/ceramic insert 14 or a polymeric insert 16, which may be used alternatively in shell 12. In other words, assembly 10 allows a surgeon to utilize a common shell portion 12 with either the two inserts 14 and 16, thereby providing a convenient assembly for implantation into a patient. Of course, a surgeon may only implant either metallic/ceramic insert 14 or polymeric insert 16 within shell portion 12 at a given time. It is noted that metal/ceramic insert 14 may be a metal shell itself having a ceramic bearing fixedly connected thereto, such as by press fitting. Alternatively, metallic/ceramic insert 14 may be constructed solely of metal or ceramic. Determination of whether to utilize metallic/ceramic insert 14 or polymeric insert 16 may depend on several factors relating to the patient's age, activity level and/or anatomy, and is best left up to the discretion of the surgeon performing the surgery. Often times, this determination is made pre-operation, but may also be made during the procedure. In either case, the aforementioned inserts are preferably adapted to receive and cooperate with the ball of a femoral head replacement. As such, inserts and femoral head replacements may be offered in combinations.

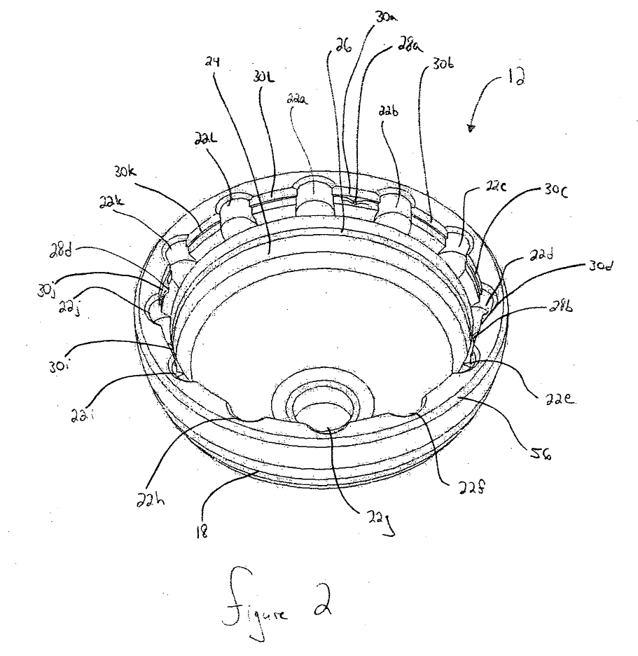

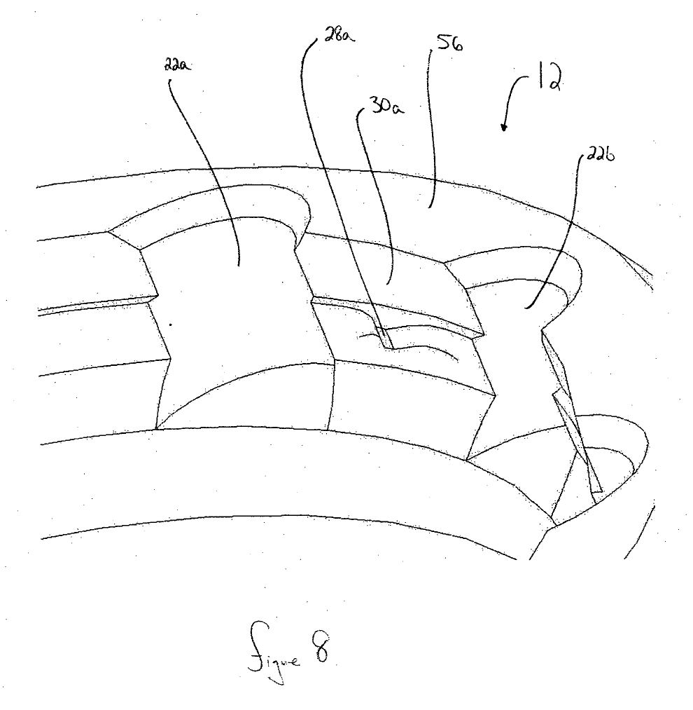

Shell portion 12 is depicted from its open end in FIG. 2. In certain embodiments, shell portion 12 may be constructed of various metals, including biocompatible metals such as cobalt chrome alloy, stainless steel and titanium. In the embodiment shown in the figures, shell portion 12 includes an outer surface 18 and an inner surface 20. Outer surface 18 is preferably sized and configured to be implanted within a previously prepared acetabulum, and may include bone growth inducing surfaces. Such preparation of the acetabulum is well known to those of ordinary skill in the art. On the contrary, inner surface 20 is preferably sized and configured to receive the aforementioned inserts 14 and 16, and includes several elements for mating/locking with the inserts. More particularly, inner or interior surface 20 of the preferred shell 12 includes twelve recesses, dimples or scallops 22a-l, a female taper 24, a circumferential groove 26, barbs 28a-d (shown in more detail in FIG. 8), and diametrical recesses 30a-l. Scallops 22a-l may be rounded recesses which are adapted to cooperate with similar rounded projections or lobes. Female taper 24 is essentially a sloped side wall as is well known in the art, such as a “Morse” taper. Groove 26 may extend around an inner circumference of shell 12 and is preferably sized and configured to receive a corresponding projection, such as an element of polymeric insert 16. Four barbs 28a-d may be relatively sharp projections designed to cut into certain material, such as polymeric material. Finally, diametrical recesses 30a-l are recessed areas located between scallops 22a-l, which create a stepped configuration of the walls between the scallops. Each of the above elements will be discussed more fully below with regard to their cooperation with the aforementioned inserts 14 and 16.

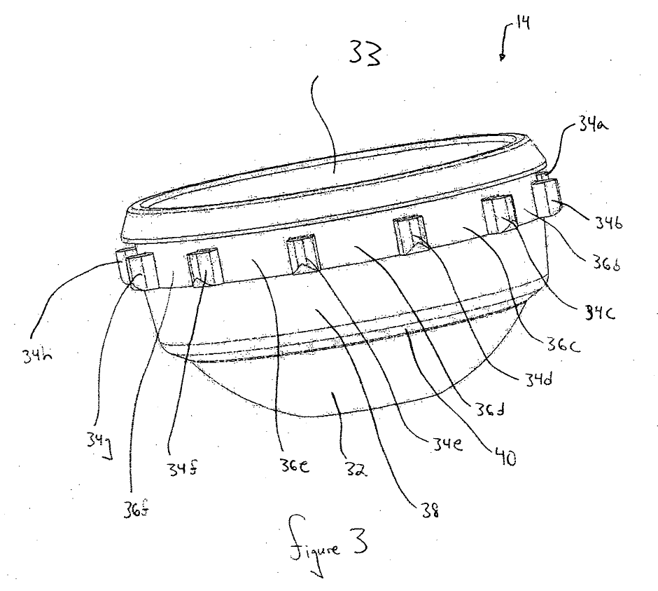

As is best shown in FIG. 3, metallic/ceramic insert 14 includes an outer portion 32 and an inner portion 33. In one preferred embodiment, like that shown in the figures and mentioned above, outer portion 32 is constructed of a biocompatible material, such as stainless steel or titanium. Contrarily, inner portion 33 may be constructed of a ceramic material or the like. Alternatively, providing an entirely metal or ceramic insert 14 is contemplated. As is briefly mentioned above, inner portion 33 may be sized and configured to mate with the ball portion of a femoral head replacement component. Thus, the surface of inner portion 34 should be such that proper motion can be restored and/or retained in the hip joint. Like inner surface 20 of shell 12, outer portion 32 may include several elements for mating/locking with the shell. Specifically, the outer surface of outer portion 32 may have twelve projections or lobes 34a-l for cooperation with the above discussed scallops 22a-l, rim portions 36a-l for use in extraction of insert 14 from shell 12 (e.g. —by providing a gap which allows for an instrument to pry insert 14 from shell 12), a male taper 38 for cooperation with the above mentioned female taper 24 of shell 12, and a centralizing chamfer 40 to aid in the alignment of insert 14 with respect to shell 12. Once again, each of the elements of insert 14 will be discussed more fully below in the discussion relating to the mating of shell 12 and insert 14.

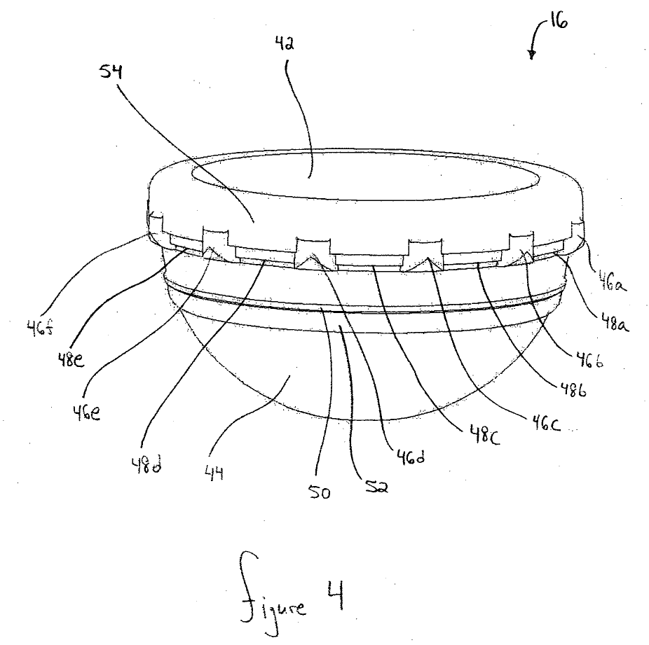

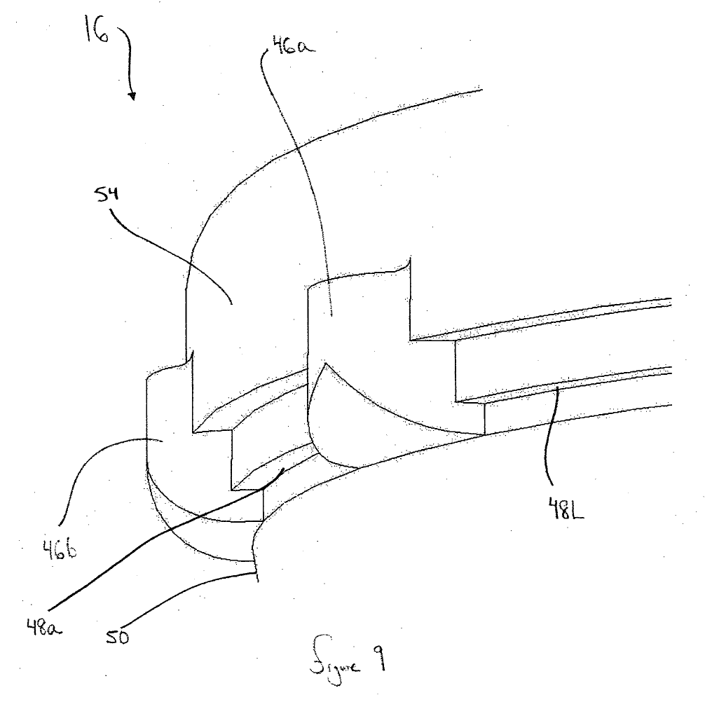

FIG. 4 depicts the remaining component of assembly 10, polymeric insert 16. Like metallic/ceramic insert 14, insert 16 includes an inner surface 42 for cooperation with the head of a corresponding femoral replacement component. Once again, inner surface 42 should be sized and configured so as to restore/allow proper motion in the hip joint to be replaced. However, unlike metallic/ceramic insert 14, insert 16 is a single pieced element, with an outer surface 44 having several elements necessary for mating and locking with shell 12. Specifically, outer surface 44 may include twelve projections or lobes 46a-l, twelve stepped sections 48a-l (shown in more detail in FIG. 9) located between lobes 46a-l, a circumferential bead 50 extending around a portion of the outer surface, a centralizing chamfer 52 to aid in the alignment of insert 16 with respect to shell 12, and a circumferential rim portion 54 for further locking of insert 16 into shell 12. As with the above discussed components of assembly 10, the elements of polymeric insert 16 will be better understood when taken in conjunction with the below description of the cooperation between shell 12 and insert 16.

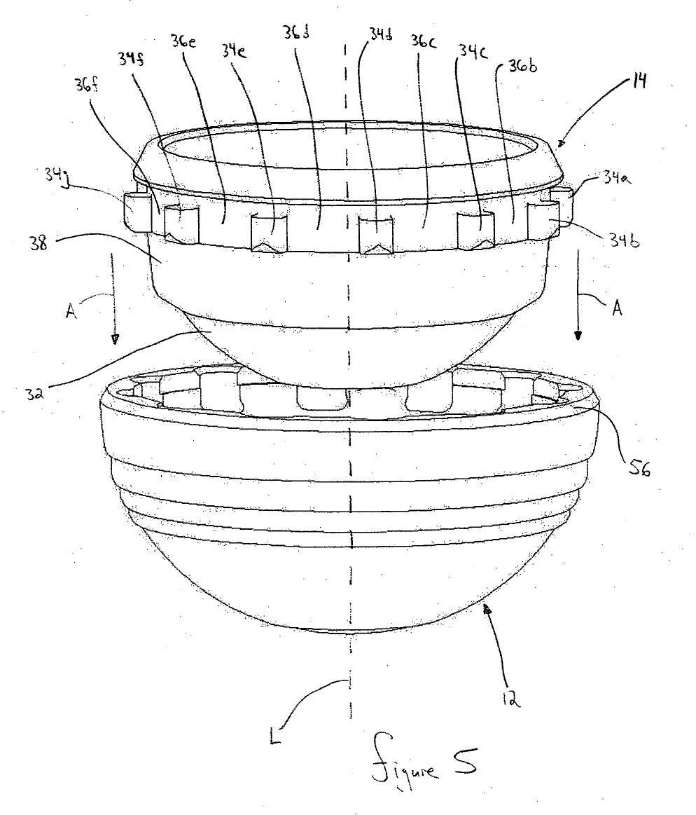

FIGS. 5-7 depict three separate methods of operatively connecting shell 12 and metallic/ceramic insert 14. Preferably, this connection is such that the two components are substantially locked together, so as to prevent most, if not all, movement between the two. As was alluded to above, the basic locking feature between shell 12 and insert 14 may be a standard taper lock between female taper 24 of shell 12 and male taper 38 of insert 14. This type of locking between two substantially circular or conical elements is well known in the engineering arts, and has been utilized in previously acetabular cup assemblies. However, it is a potential problem in previous incarnations of acetabular cup assemblies that the shell and insert become canted or misaligned during insertion of the insert into the shell. Although basic engineering principles dictate that taper locks should preferably include a length of engagement which is equal to twice the taper diameter, in order to ensure proper alignment, such is not possible in the relatively short maximum section of taper that can exist in an acetabular cup design. Thus, the above noted canting problem may occur, thereby leaving an insert locked into an angled position with respect to the shell. In fact, one way to ensure that the inserts and shells of previous acetabular cup replacements become properly engaged with one another is to align the respective axis of the two components and thereafter insert the insert purely by movement in the direction of the aligned axis. However, this is often not possible within the relatively small constraints of a hip replacement surgery, especially one conducted in accordance with minimally invasive surgery (MIS) techniques. Such a small operating space also often prevents a surgeon from visually inspecting the connection between the shell and insert. Acetabular cup assembly 10 of the present invention is designed so as to prevent the above problems associated with canting or misalignment of shell 12 and insert 14. More particularly, the cooperation between scallops 22a-l and lobes 34a-l of insert 14 ensures proper alignment of the two components, which ultimately results in proper locking therebetween.

As mentioned above, shell 12 and insert 14 can be attached together in three different fashions. The first of these attachment methods is illustrated in FIG. 5, where insert 14 is essentially aligned axially with shell 12 along an axis L. This method is like the aforementioned prior art methods of connecting inserts with implanted shells, and as discussed above, may not be possible in relatively small operating spaces. Nevertheless, it is indeed one method of attaching insert 14 to shell 12. As shown in FIG. 5, subsequent to being axially aligned with shell 12, insert 14 is placed into the shell in the direction of arrow A. In this position, lobes 34a-l either align with scallops 22a-l, or the lobes rest on the top surface 56 (shown in FIGS. 2 and 5) of shell 12. In the latter case, a rotation of insert 14 is required in order to have lobes 34a-l and scallops 22a-l align. Given the fact that in the preferred embodiment twelve lobes and scallops exist on the respective insert 14 and shell 12, the maximum amount of rotation required to align the lobes and scallops will be approximately 15 degrees. Once lobes 34a-l and scallops 22a-l are properly aligned, insert 14 may be locked into shell 12 with a couple of swift blows using an impaction tool (not shown). This operation essentially forces male taper 38 into female taper 24, and keeps shell 12 and insert 14 from becoming dislodged from one another absent certain specific forces being applied thereto. Centralizing chamfer 40, although primary shaped to soften the edges of insert 14, may also aid in keeping insert 14 properly aligned with shell 12 during this insertion procedure. In the fully assembled state, the chamfer is designed to provide clearance with the bottom of shell 12, and may further limit canting of the insert with respect to the shell.

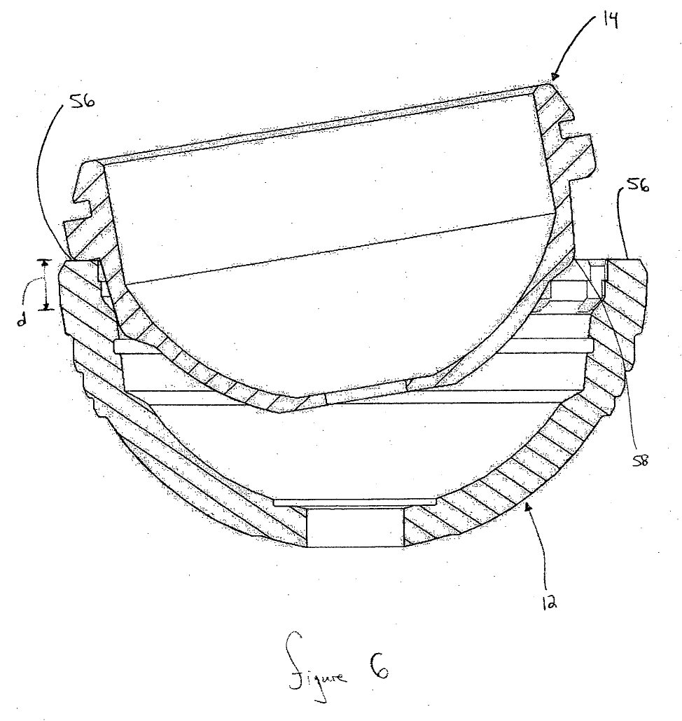

The second method of attaching shell 12 and insert 14, where the two parts are initially misaligned, is depicted in FIG. 6. In this method, insert 14 is first arranged so that some of lobes 34a-l initially rest on top of top surface 56 of shell 12. A distance d between the start of female taper 24 and top surface 56 is preferably such that a trailing edge 58 of male taper 38 of insert 14 always clears the remainder of the geometry of shell 12. This allows for insert 14 to naturally establish coaxial alignment with shell 12, upon further insertion of trailing edge 58 into shell 12. In other words, distance d allows for portions of insert 14 to be placed atop shell 12 at any angle and the remainder of the insert to be swung into the shell. This may allow a surgeon to connect shell 12 and insert 14 together without having to align them first axially. Once trailing edge 58 is completely inserted into shell 12, lobes 34a-l rest on the top surface 56, as discussed above in relation to the first method of inserting insert 14 into shell 12. Similar to the above first method, this position requires a maximum 15 degree rotation of insert 14 with respect to shell 12, in order to have lobes 34a-l align with scallops 22a-l. Thereafter, an impaction tool or the like may be utilized to force male taper 38 into female taper 24, as is also discussed above. Once again, centralizing chamfer 40 may aid in keeping insert 14 properly aligned with shell 12.

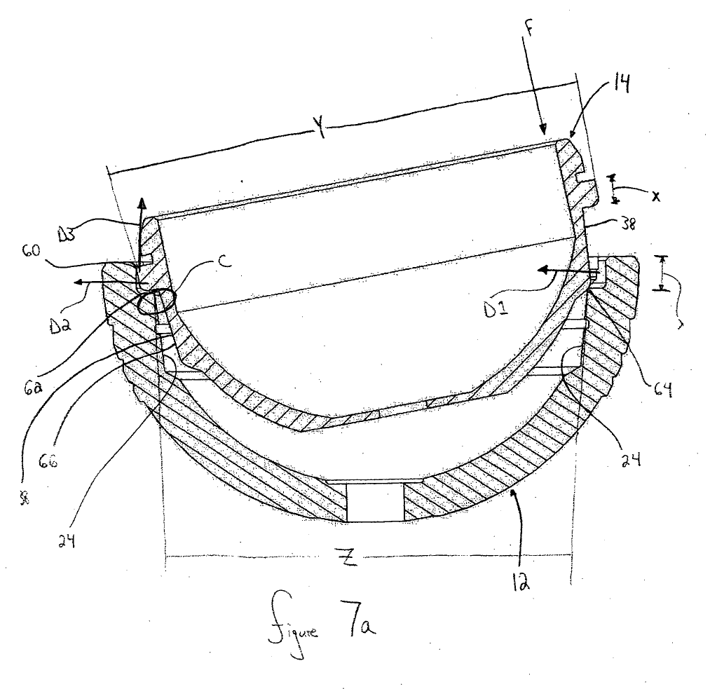

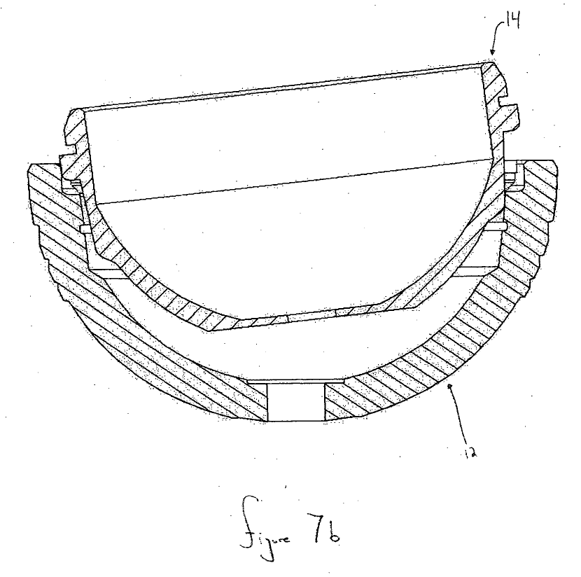

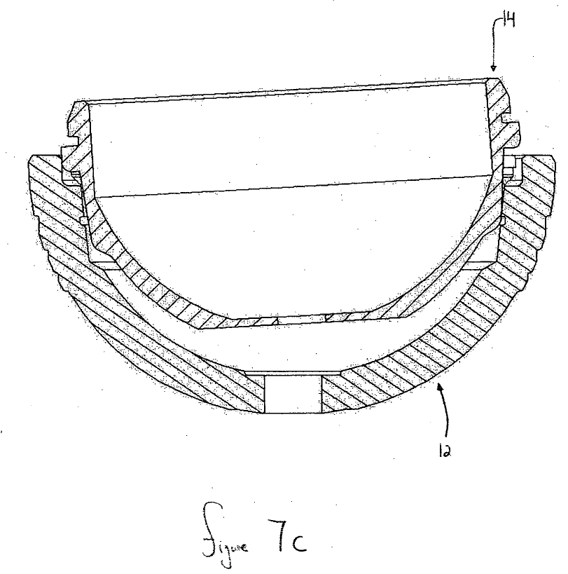

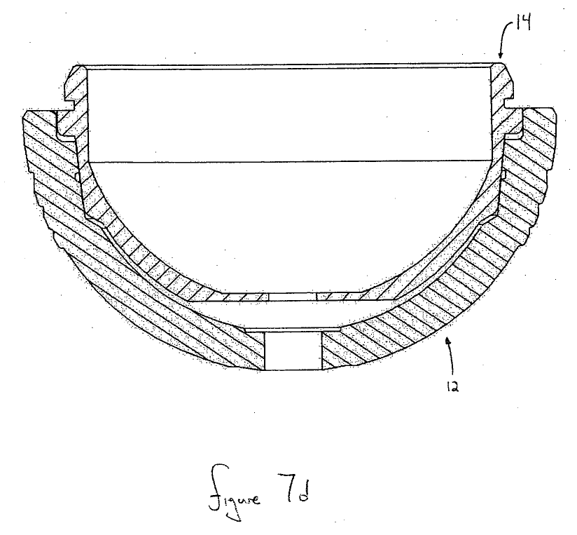

The third method of attaching a misaligned shell 12 and insert 14 is depicted in FIGS. 7a-7d. The major difference between this third method and that of the second method is that some of lobes 34a-l are initially aligned and inserted into scallops 22a-l. In this position, insert 14 and shell 12 are initially in contact at points 60, 62 and 64, and there preferably exists a clearance between a leading edge 66 of male taper 38 and an adjacent portion of female taper 24 (the clearance being depicted by the circled section C). This clearance is formed because of the ratio between a height X of each lobe 34a-l and a diameter Y, both of insert 14, and a diameter Z of female taper 24 of shell 12. Each of these dimensions is best depicted in FIG. 7a. In addition, it is noted that each scallop 22a-l may have a depth X′, which is preferably slightly larger than height X of each lobe 34a-l. This may also play into the formation of clearance C. It is noted that other factors, such as tolerances taper angles, fillet sizes, etc., may also affect the relationship between insert 14 and shell 12 and the formation of clearance C. Nonetheless, in one preferred embodiment, the relationship between X, Y and Z is such that the following equation applies:
R*(Y−Z)/2=X
where R is between 1.4 and 1.6

However, the above equation is merely one such equation for providing the proper relationship between shell 12 and insert 14. For example, another equation for providing the proper relationship between X, Y and Z could read as follows:
1.33X=(Y−Z)
(i.e. —where R is 1.5)

Given the above discussion relating to the relationship between shell 12 and insert 14, it is to be understood that the two components can be assembled together from the position shown in FIG. 7a. Upon an application of a force F, insert 14 preferably moves in a cam action in the directions D1, D2 and D3. These directions are all depicted in FIG. 7a, and the movement of insert 14 with respect to shell 12 is depicted in FIGS. 7b-7d. Thus, upon the application of force F, insert 14 naturally establishes coaxial alignment with shell 12 (shown in FIGS. 7b-7c), whereupon insert 14 may be seated within shell 12 (shown in FIG. 7d) through the utilization of the above mentioned impaction tool and method of using same. The aforementioned second and third methods of attaching shell 12 and insert 14 are particularly useful for use in relatively small operating spaces, such as those typically present in MIS procedures. In addition, the inclusion a scallops 22a-l and lobes 34a-l allows a surgeon to be sure that insert 14 has been properly seated within shell 12, without visually confirming same. There simply is no possibility of insert 14 becoming implanted in a canted fashion within shell 12, because of these aligning aids.

With regard to the attachment of shell 12 and polymeric insert 16, it is first noted that assembly 10 of the present invention is designed so as to allow insert 16 to be inserted into shell 12 in any of the same fashions described above in connection with insert 14. Thus, insert 16 may be initially positioned in vertical alignment with shells 12, at an angle such that some of lobes 46a-l of insert 16 rest on top surface 56 of shell 12, or at angle such that some of lobes 46a-l are aligned and inserted into scallops 22a-l. Once again, this provides a surgeon with the opportunity to work in relatively small operation areas, such as those present during MIS procedures. Although the initial insertion of polymeric insert 16 into shell 12 may be done in a similar fashion to that of metallic/ceramic insert 14, the ultimate seating of insert 16 is quite different. However, like that of insert 14, the connection between shell 12 and insert 16 is preferably such that rotational and/or vertical or push out movements, as well as micro-movements are prevented between the two components. Therefore, both inserts 14 and 16 are preferably capable of being inserted either axially or at an angle between their axis, and are prevented from substantially all movement with respect to shell 12.

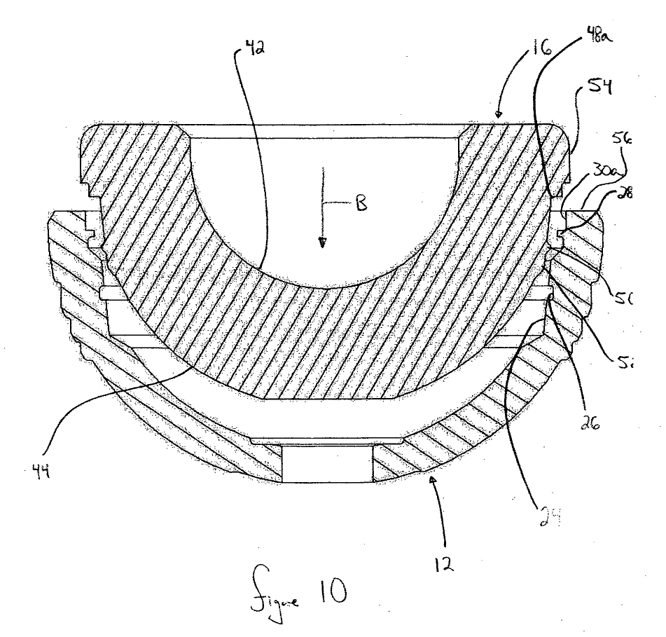

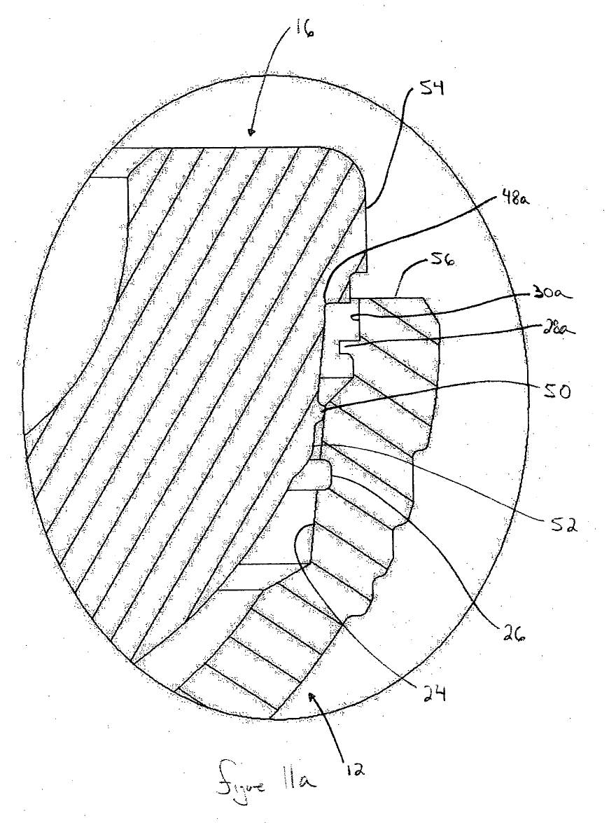

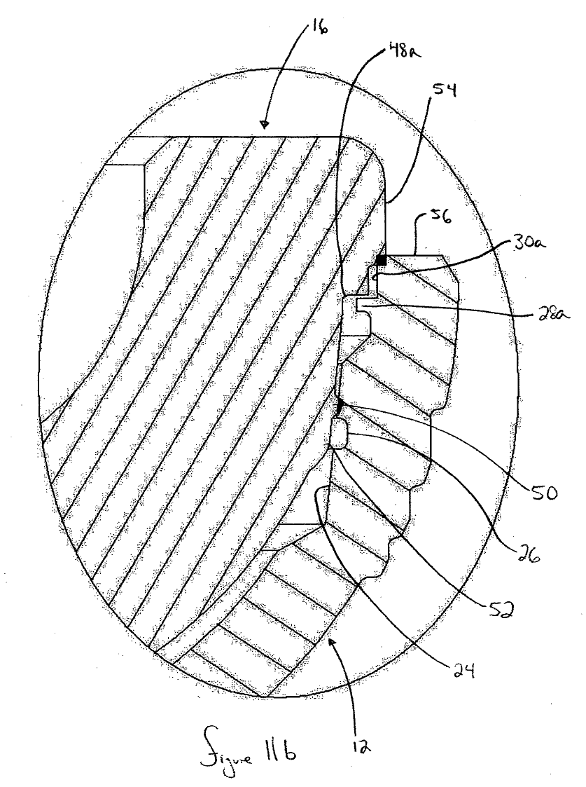

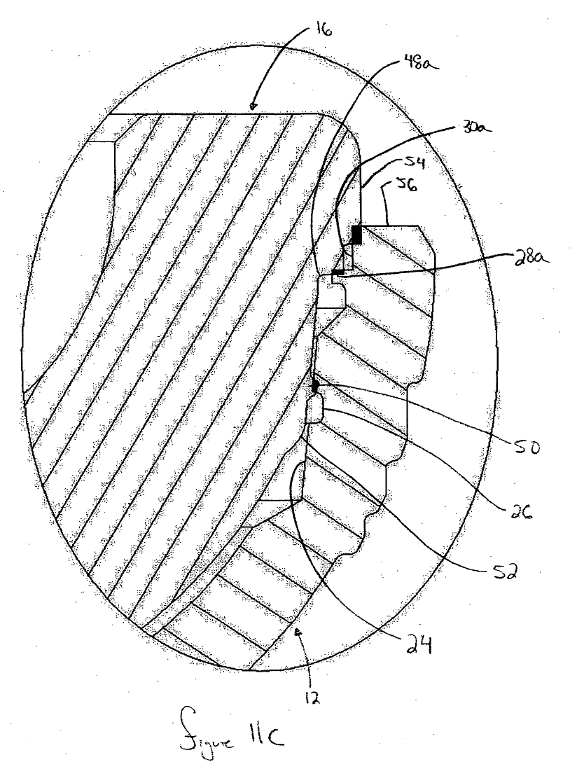

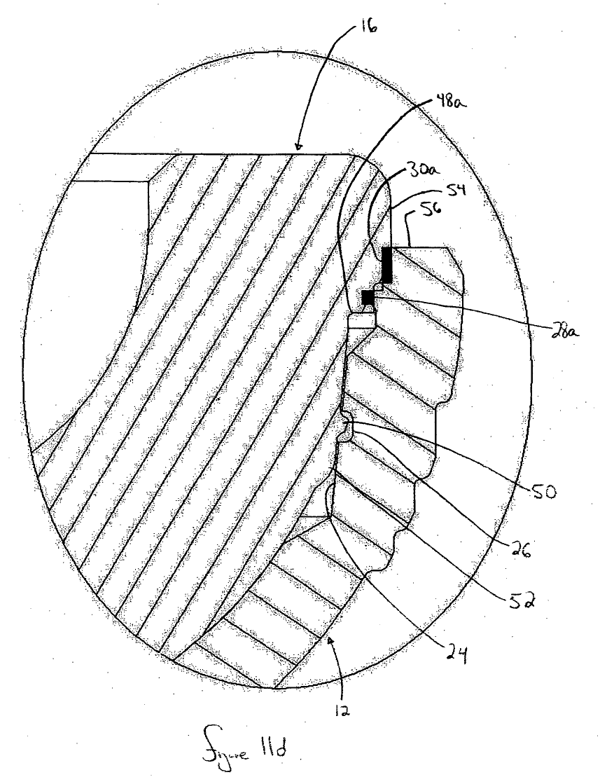

To achieve this cooperation and attachment between shell 12 and insert 16, assembly 10 of the present invention utilizes a series of sequentially stepped locking features. More particularly, connection between shell 12 and insert 16 is such that the above mentioned elements of each component are designed so as to achieve proper constraints while continuing to be easy to assembly. FIG. 10 depicts shell 12 with insert 16 in an aligned position during the assembly process. This position is similar to the naturally established coaxial alignment discussed above in relation to the third method of attaching insert 14 to shell 12. It is noted that centralizing chamfer 52 may aid in this alignment, as described above. However, it is noted that the insert may at least initially be aligned in different fashions, including axial alignment or an angled nature. From the position depicted in FIG. 10, the next step is to apply a force from an impaction tool (not shown) in order to put insert 16 in its final locked position within shell 12. During this application of force, the locking mechanisms between the two components go through several sequential steps. FIGS. 11a-d depict these steps during the attachment procedure.

First, upon application of a force by the aforementioned impaction tool, insert 16 moves in a direction depicted by arrow B in FIG. 10 and lobes 46a-l of insert 16 engage scallops 22a-l of shell 12 to establish alignment and ensure concentricity between the two components. It is noted that a rotation, similar to that discussed above in the discussion on insert 14, may be required to initially properly align insert 16 and shell 12. Once again, the maximum amount of rotation should be no more than approximately 15 degrees. Upon further application of a force, as is best shown in FIG. 11b, insert 16 continues to move in the direction of arrow B and circumferential bead 50 begins to hit female taper 24 of shell 12, thereby beginning to compress the bead. At the same time, and continuing upon further application of a force from the impaction tool, circumferential rim 54 of insert 16 begins to engage recess 30a so as to compress at least a portion of the insert to thereby form an interference fit between the two components. This is also seen in FIG. 11b, and continues in FIGS. 11c and 11d.

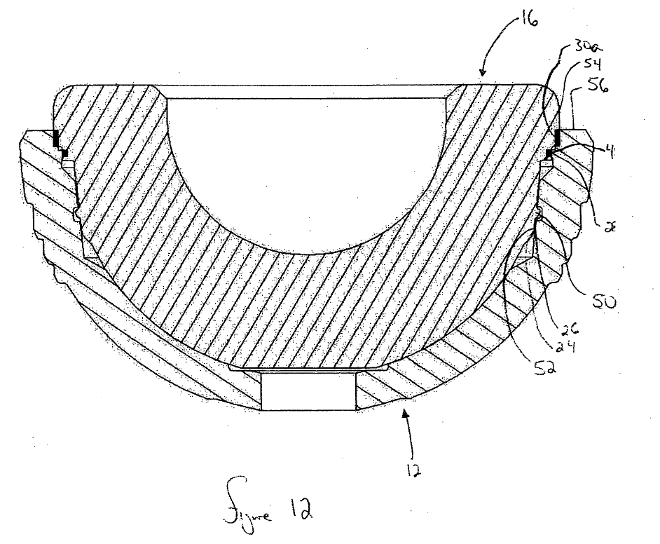

It is noted that application of a force from an impaction tool or the like may require the surgeon to operate the tool in intervals. In other words, the tool may act like a standard hammer, where successive engagements with insert 16 may further push the insert into shell 12. Beginning in FIG. 11c, the aforementioned four barbs 28a-d (only barb 28a is depicted in FIGS. 11a-d) start to cut into the wider section of stepped sections 48a-l aligned therewith (only section 48a is depicted in FIGS. 11a-d). This engagement creates yet another interference fit which aids in the prevention of micro-movements between insert 16 and shell 12. It is noted that relationship between the barbs and the stepped sections is preferably such that the barbs only engage the wider section of the stepped section. This should also result in the barbs not engaging any portion of metal insert 14. Finally, as shown in FIG. 11d, once insert 16 is fully inserted into shell 12, circumferential bead 50 slips into groove 26 in a well known manner, and the previously compressed bead 50 expands to establish a primary locking device. This locking device prevents vertical movement of insert 16 with respect to shell 12. In the position shown in FIG. 11d, and more fully depicted in FIG. 12, insert 16 is fully seated within shell 12. Essentially, the spherical diameters of the two components substantially match so as to allow this seating.

It is noted that the embodiment depicted in the figured and discussed in this detailed description is merely one such embodiment in accordance with the present invention. As such, variations of any of the components of assembly 10 may fall within the scope of the present invention. For example, although specifically sized and shaped components and elements are shown and described in the present application, such components and elements may vary in size and shape. In one instance, it is contemplated to provide differently shaped lobes and corresponding scallops. Specifically, rather than the rounded lobes and scallops shown in the drawings, it is noted that rectangular lobes and scallops can be utilized in accordance with shell 12 and either insert 14 or 16 of the present invention. In addition, although specific materials for constructing assembly 10 are discussed herein, many different types of materials can be utilized. For example, insert 16 is described herein as being constructed of a polymeric material, such as Ultra High Molecular Weight Polyethylene. However, given the operation of insert 16 and its cooperation with shell 12, it is contemplated to provide any relatively flexible material to construct the insert.

Although not specifically set forth herein, it is to be understood that assembly 10 of the present invention may be utilized during any type of hip surgery known in the art. This includes total hip surgery methods performed prior to the time of the present application, as well as those performed in the future. Typically, these types of surgeries entail accessing the hip joint of a patient, resecting the end of the femur and replacing such with a femoral replacement prosthesis, and resurfacing the acetabulum. Assembly 10 of the present invention is preferably utilized in conjunction with this resurfaced acetabulum. Initially, shell 12 is implanted in the resurfaced acetabulum, and either insert 14 or 16 is thereafter inserted within and attached to shell 12 in accordance with the above descriptions. In this regard, it is noted that kits may be provided for allowing a surgeon to choose differently sized shells 12 and corresponding inserts 14 or 16. The required size of shell 12 may differ depending upon the anatomy of the patient and/or the amount of damage to the acetabulum, as well as many other factors.

Finally, as the joints of the hip and shoulder are substantially similar, it is contemplated to provide a replacement assembly in accordance with the present invention for use in a shoulder replacement surgery. Obviously, such an assembly would have to be sized and configured to fit within the particular shoulder anatomy of a patient.

Although the invention herein has been described with reference to particular embodiments, it is to be understood that these embodiments are merely illustrative of the the principles and applications of the present invention. It is therefore to be understood that numerous modifications may be made to the illustrative embodiments and that other arrangements may be devised without departing from the spirit and scope of the present invention as defined by the appended claims.

1. A prosthetic cup assembly comprising:
a metallic shell having an outer shell surface and an inner shell surface, the outer shell surface being adapted for insertion into the acetabulum of a patient and the inner shell surface including a plurality of scallops and a female taper; and
a metallic insert having an outer insert surface, the outer insert surface being adapted for insertion into the inner shell surface of the shell, the outer insert surface including a plurality of lobes adapted for engagement with the scallops and a male taper adapted for engagement with the female taper,
wherein the dimensional relationship between the insert and the shell is such that the insert may be inserted into the shell from an axially aligned position or from an angled position, and insertion of the outer insert surface within the inner shell surface allows fixable attachment of the insert to the shell.

2. The prosthetic cup assembly of claim 1, wherein the shell is constructed of a material selected from the group consisting of cobalt chrome, stainless steel and titanium.

3. The prosthetic cup assembly of claim 1, wherein the shell includes twelve scallops and the insert includes twelve lobes.

4. The prosthetic cup assembly of claim 3, wherein the shell further includes a circumferential groove, twelve diametrical recesses, and at least one barb.

5. The prosthetic cup assembly of claim 4, wherein the insert further includes twelve rim portions and a centralizing chamfer.

6. The prosthetic cup assembly of claim 1, wherein the outer shell surface may include bone growth inducing surfaces.

7. The prosthetic cup assembly of claim 1, wherein the insert may further include a ceramic insert adapted to cooperate with the head of a femoral replacement component.

8. The prosthetic cup assembly of claim 1, wherein the insert is inserted into the shell in situ.

9. The prosthetic cup assembly of claim 1, wherein each lobe has a height X, the insert has a diameter Y, and the shell 12 has a diameter Z.

10. The prosthetic cup assembly of claim 9, wherein the relationship between X, Y and Z is such that R*(Y−Z)/2=X, where R is between 1.4 and 1.6.

11. A prosthetic cup assembly comprising:
a metallic shell having an outer shell surface and an inner shell surface, the outer shell surface being adapted for insertion into the acetabulum of a patient and the inner shell surface including a plurality of scallops, a circumferential groove and at least one barb; and
a polymeric insert having an outer insert surface, the outer insert surface being adapted for insertion into the inner shell surface of the shell, the outer insert surface including a plurality of lobes and a circumferential bead,
wherein the plurality of lobes are adapted for engagement with the scallops, the circumferential bead is adapted for engagement with the circumferential groove, and the at least one barb is adapted for engagement with the polymeric insert upon insertion of the outer insert surface within the inner shell surface, thereby fixably attaching the insert to the shell.

12. The prosthetic cup assembly of claim 11, wherein the shell is constructed of a material selected from the group consisting of cobalt chrome, stainless steel and titanium.

13. The prosthetic cup assembly of claim 11, wherein the shell includes twelve scallops and the insert includes twelve lobes.

14. The prosthetic cup assembly of claim 13, wherein the shell further includes a female taper and twelve diametrical recesses.

15. The prosthetic cup assembly of claim 14, wherein the insert further includes a rim portion and a centralizing chamfer.

16. The prosthetic cup assembly of claim 11, wherein the outer shell surface may include bone growth inducing surfaces.

17. The prosthetic cup assembly of claim 11, wherein the insert may further include an inner surface adapted to cooperate with the head of a femoral replacement component.

18. The prosthetic cup assembly of claim 11, wherein the insert may be inserted into the shell from an axially aligned position or from an angled position.

19. A kit for use in a hip replacement surgery comprising:
at least one shell, the shell having an outer shell surface and an inner shell surface, the outer shell surface being adapted for insertion into the acetabulum of a patient and the inner shell surface including a plurality of scallops, a female taper, a circumferential groove and at least one barb;
at least one metallic insert having an outer metallic insert surface being adapted for insertion into the inner shell surface of the shell, the outer metallic insert surface including a plurality of lobes and a male taper, where the plurality of lobes are adapted for engagement with the scallops and the male taper is adapted for engagement with the female taper upon insertion of the outer insert surface within the inner shell surface, thereby fixably attaching the insert to the shell; and
at least one polymeric insert having an outer insert surface being adapted for insertion into the inner shell surface of the shell, the outer insert surface including a plurality of lobes and a circumferential bead, where the plurality of lobes are adapted for engagement with the scallops, the circumferential bead is adapted for engagement with the circumferential groove, and the at least one barb is adapted for engagement with the polymeric insert upon insertion of the outer insert surface within the inner shell surface, thereby fixably attaching the insert to the shell.

20. The kit of claim 19, wherein both the at least one metallic insert and the at least one polymeric insert may be inserted into the shell from an axially aligned position or from an angled position.

21. The kit of claim 19, wherein the metallic insert may further include a ceramic insert adapted to cooperate with the head of a femoral replacement component.

22. The kit of claim 19, further including at least one femoral replacement component.

23. The prosthetic cup assembly of claim 11, wherein the insert further includes a plurality of stepped sections.

24. The prosthetic cup assembly of claim 23, wherein the shell includes a plurality of barbs adapted for engagement with the stepped sections.

25. The kit of claim 19, wherein the insert further includes a plurality of stepped sections.

26. The kit of claim 25, wherein the shell includes a plurality of barbs adapted for engagement with the stepped sections.
